(19) United States
(12) Patent Application Publication
(10) Pub. No.: US 2023/0414715 A1
(43) Pub. Date: Dec. 28, 2023

(54) TRAIL COMPOUNDS FOR REDUCING CANCER TREAMENT SIDE EFFECTS

(71) Applicant: BROWN UNIVERSITY, Providence, RI (US)

(72) Inventors: Wafik S. El-Deiry, Barrington, RI (US); Anna Louie, Providence, RI (US); Jillian Strandberg, Vernon, CT (US)

(21) Appl. No.: 18/327,193

(22) Filed: Jun. 1, 2023

Related U.S. Application Data
(60) Provisional application No. 63/347,619, filed on Jun. 1, 2022.

(51) Int. Cl. A61K 38/17; A61K 31/519; A61P 11/00

(52) U.S. Cl. CPC A61K 38/1774; A61K 31/519; A61P 11/00

(57) ABSTRACT

A method of reducing toxic side effects in a subject undergoing cancer treatment is described. The method includes administering an effective amount of a TRAIL compound to the subject.

### STATEMENT REGARDING FEDERALLY SPONSORED RESEARCH

This invention was made with government support under Grant Number CA173453 awarded by the National Institutes of Health. The Government has certain rights in this invention.

### CROSS-REFERENCE TO RELATED APPLICATIONS

This application claims priority to U.S. Provisional Application Ser. No. 63/347,619, filed on Jun. 1, 2022, which is hereby incorporated by reference in its entirety.

### BACKGROUND

Cancer treatment is known to often involve a number of undesirable toxic side effects. For example, radiation therapy is a curative treatment for many malignancies and provides effective palliation in patients with tumor-related symptoms. However, the biophysical effects of radiation therapy are not specific to tumor cells and may produce toxicity due to exposure of surrounding organs and tissues. Wang, K., Tepper, J., CA Cancer J Clin., 71(5):437-454 (2021). The acute and chronic effects of radiation toxicity to the lungs and other organs involve inflammation and fibrosis, and are debilitating. These undesirable effects typically occur within weeks to months of exposure to ionizing radiation.

Preclinical data support the potential of the death-signaling receptors for TRAIL as targets for cancer therapy. However, it is unclear whether these death-signaling receptors suppress the emergence and growth of malignant tumors in vivo. The inventors have shown that TNF-related apoptosis-inducing ligand receptor (TRAIL-R), the only proapoptotic death-signaling receptor for TRAIL in the mouse, suppresses inflammation and tumorigenesis. Grosse-Wilde et al., J Clin Invest., 118(1):100-10 (2008). Loss of a single TRAIL-R allele on the lymphoma-prone Eμ-myc genetic background significantly reduced median lymphoma-free survival. TRAIL-R—deficient lymphomas developed with equal frequency irrespective of mono- or biallelic loss of TRAIL-R, had increased metastatic potential, and showed apoptotic defects relative to WT littermates. In addition, TRAIL-R^{-/-} mice showed decreased long-term survival following a sublethal dose of ionizing radiation. Histological evaluation of moribund irradiated TRAIL-R^{-/-} animals showed hallmarks of bronchopneumonia as well as tumor formation with increased NF-κB p65 expression. TRAIL-R also suppressed diethylnitrosamine-induced (DEN-induced) hepatocarcinogenesis, as an increased number of large tumors with apoptotic defects developed in the livers of DEN-treated TRAIL-R^{-/-} mice. Thus TRAIL-R may function as an inflammation and tumor suppressor in multiple tissues in vivo.

### SUMMARY

Cancer therapy is limited by toxicity from pneumonitis, lung fibrosis and exacerbated by innate immune death receptor DR5-deletion in mice. Protection from therapy-induced lung injury is a long-term goal in oncology. We investigated whether boosting innate immune signaling might protect from pneumonitis. Parenteral PEGylated trimeric TRAIL (TLY012) and oral TRAILInducing Compound #10 (TIC10/ONC201) protect mice from 20 Gy radiation-induced pneumonitis and fibrosis with 2-weeks of treatment administered at exposure. Complete protection from lethality, reduced alveolar-wall thickness, lessened inflammation and improved oxygen-saturation are observed in radiation-exposed wild-type (WT) and TRAIL-/- C57B1/6 mice. There is less fibrosis at 22-weeks in TLY012-rescued survivors vs un-rescued surviving irradiated mice. WT-mice bearing orthotopic breast tumors receiving 20 Gy thoracic radiation dose are protected from pneumonitis with disappearance of tumors. Reduced CCL22/MDC levels occur in TLY012-treated, irradiated mice. Modulation of TRAIL/DR5 signaling prevents pneumonitis after radiation exposure and has implications for lung injury in general.

### BRIEF DESCRIPTION OF THE FIGURES

FIG. 1A, FIG. 1B and FIG. 1C provide graphs and images showing the experimental design including mouse strains and treatment cohorts A) Hypothesized outcomes regarding lung protection of C57B1/6, DR-5-/-, and TRAIL-/- mice irradiated with a whole-thorax x-ray irradiation dose of 20 Gy and treatment of either 100 mg/kg of ONC201 weekly, 10 mg/kg of TLY012 twice a week, or no treatment. B) Preliminary experimental timeline where mice received their first treatment just before radiation, and then were treated with either ONC201 once a week or TLY012 twice a week and then sacrificed on day 13 post-irradiation. C) First observations of suppression of radiation pneumonitis seen in ×20 H&E-stained lung tissue of male mice by short term treatment with TRAIL pathway agonists as indicated (n=2/treatment/genotype).

FIG. 2A and FIG. 2B provide images showing protection of mice from radiation pneumonitis depends on genetic strain and TRAIL pathway agonist used to investigate mechanism of radioprotection. A,B) H&E stains of lung tissue from male and female mice from C57B1/6, DR5-/-, and TRAIL-/- backgrounds treated with either ONC201, TLY012, or control gavage (n=2/gender/genotype/treatment) 13 days post-irradiation (top row ×4, bottom row ×40) scale bar: 100 μm.

FIG. 3A, FIG. 3B and FIG. 3C provide graphs showing protection for chest-irradiation induced mouse lethality by TLY012. A) Survival of male TRAIL-/- mice following 18 Gy irradiation led to an increased survival in mice treated with 10 mg/kg of TLY012 at 10 weeks following whole-thorax irradiation (n=5/treatment/group). B) Survival of female TRAIL-/- mice following 18 Gy whole-thorax irradiation was increased to 3 weeks in mice treated with TLY012 compared to control mice that survived 2 weeks after radiation (n=2/treatment/group). C) Survival of female C57B1/6 mice was increased by approximately a week in mice treated with TLY012 compared to control mice (n=6/treatment/group).

FIG. 4 provides images of a Masson's Trichrome stain of male TRAIL-/- mouse lung 22 weeks post-radiation show rescue in TLY012 treated mice. A) Male TRAIL-/- mice that were treated with a single thoracic x-ray irradiation dose of 18 Gy and were treated with TLY012 or remained as control for 22 weeks post-radiation (n=3, n=2). Lung tissue was stained with Masson' s Trichrome and imaged at ×10. (red=muscle fibers, bright blue=collagen, dark red/blue=nuclei).

FIG. 5A, FIG. 5B and FIG. 5C provide graphs showing Nanostring nCounter PanCancer Immune panel for TRAIL-/- mice with and without rescue from radiation injury by TRAIL agonist. A) Volcano plot of all 40 references genes and their fold change compared to control mice. B) Heat map of log2 fold change of the top 16 significantly different genes between control and TLY012 treatment (n=6). C) Heat map of serum cytokine level fold change of TLY012 treated mice relative to untreated control mice.

FIG. 6A, FIG. 6B, FIG. 6C, FIG. 6D and FIG. 6E provide graphs showing the efficacy of chest irradiation using an orthotopic breast cancer model while preventing pneumonitis by TLY012 and reducing CCL22. A) Experimental timeline of female C57B1/6 mice orthotopically injected with e0771 on day 0 and when tumors reached 2-5 mm in size mice were irradiated with whole-thorax irradiation dose of 20 Gy and treated with either TLY012, ONC201, or a combination of both (n=3/treatment/group). B,C) Tumors were removed after mice were euthanized 18 days post-cell injection where weight and volume were calculated. D) pulse oximetry readings pre-radiation and 9 days-post radiation showed oxygen saturation was more conserved in the TLY012 treated group compared to the irradiated control group. E) H&E stains of lung tissue 9 days-post irradiation showed a reduction in alveolar-wall thickness and decreased inflammation in mice treated with TLY012. Statistical analysis of cytokine fold change showed a significant decrease in levels of MDC/CCL22 (p=0.035) when compared to the irradiated control group.

FIG. 7A and FIG. 7B provide images showing respiration-gated imaging in mice irradiated with whole-thorax x-ray irradiation dose of 15 Gy with and without TLY012 treatment. A) Representative μCT images of mouse lungs were unirradiated, irradiated with 15 Gy, or irradiated with 15 Gy and rescued with TLY012 treatment during inhale duration of the breathing cycle (n=2/group). Mouse weights were 19.6 g, 24.1 g, and 22.4 g respectively. B) 3D reconstruction of mouse lungs from μCT images during exhale and inhale portions of the breathing cycle. All images are subjected to a 7% opacity filter in CTVol software.

### DETAILED DESCRIPTION OF THE INVENTION

The present invention provides a method of reducing toxic side effects in a subject undergoing cancer treatment. The method includes administering an effective amount of a TRAIL compound to the subject.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction or differentiation of the undesirable proliferating cells with minimal effects on normal cells. In accordance with the present invention, desired mechanisms of treatment at the cellular level include stimulation of differentiation in cancer and pre-cancer cells.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the inhibition of cancer growth by a detectable amount.

The term “effective amount” as used herein refers to an amount sufficient to achieve an intended result. An “effective amount” includes an amount that is 100% effective in achieving that result, but also includes amounts that are less effective but still exhibit a significant effect. For example, an effective amount of a compound for reducing toxic side effects is an amount sufficient to reduce, but not necessarily eliminate, those effects.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

### Reducing Toxic Side Effects using TRAIL Compounds

In one aspect, the invention provides a method of reducing toxic side effects in a subject undergoing cancer treatment, comprising administering an effective amount of a TRAIL compound to the subject.

The present invention includes the step of treating cancer in a subject. Cancer is a disease of abnormal and excessive cell proliferation. Cancer is generally initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of cancer cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. In some embodiments, the cancer is selected from the group of cancer types consisting of sarcoma, carcinoma, and lymphoma.

Cancer can also be characterized based on the organ in which it is growing. Examples of cancer characterized in this fashion include bladder cancer, prostate cancer, liver cancer, breast cancer, colon cancer, and leukemia. Solid tumors are more associated with the formation of an immune suppressive tumor microenvironment. In some embodiments, the cancer being treated a solid tumor cancer selected from the group consisting of breast, colon, bladder, prostate, and lung cancer.

The type of cancer present in a subject can be determined using diagnostic methods known to those skilled in the art. Common tests used to help diagnose different types of cancer include blood chemistry tests, complete blood counts, cytogenetic analysis, immunophenotyping, liquid biopsy, sputum cytology, tumor marker tests, urinalysis, and various imaging methods such as CT scan, MRI, ultrasound, and PET scan. In some embodiments, the subject has been diagnosed as having lung cancer. See Shim et al., Fam Pract., 31(2):137-48 (2013) for a review of lung cancer diagnosis. Lung cancer is typically diagnosed using a chest X-ray followed by a biopsy of suspected tissue.

A wide variety of methods are known for cancer treatment, which vary depending on the type and stage of the cancer being treated. Methods of cancer treatment include radiation therapy, chemotherapy, radiofrequency ablation, cryoablation, thermal ablation, electroporation, alcohol ablation, high intensity focused ultrasound, photodynamic therapy, chimeric antigen receptor (CAR) T-Cell therapy, administration of monoclonal antibodies, and administration of immunotoxins. Cancer treatment can be used for both prophylactic and therapeutic treatment.

In some embodiments, the cancer treatment is radiation treatment. Radiation therapy or radiotherapy, often abbreviated RT, RTx, or XRT, is a cancer therapy using ionizing radiation delivered by a linear accelerator. Ionizing radiation works by damaging the DNA of cancerous tissue leading to cellular death. Radiation therapy may be used to treat a number of types of cancer so long as they are localized to one area of the body. It may also be used as part of adjuvant therapy, to prevent tumor recurrence after surgery to remove a primary malignant tumor. To spare normal tissues (such as skin or organs which radiation must pass through to treat the tumor), shaped radiation beams are aimed from several angles of exposure to intersect at the tumor, providing a much larger absorbed dose there than in the surrounding healthy tissue. For curative cases, the typical dose for a solid epithelial tumor ranges from 60 to 80 grays (Gy), while lymphomas are treated with 20 to 40 Gy, while preventive doses are typically around 45-60 Gy.

Many common, moderately radioresponsive tumors are routinely treated with doses of radiation therapy, particularly if they are at an early stage. Examples of cancer that can be treated in this way include non-melanoma skin cancer, head and neck cancer, breast cancer, non-small cell lung cancer, cervical cancer, anal cancer, and prostate cancer. In some embodiments, the cancer being treated is lung cancer. Lung cancer can be treated using 45 Gy doses twice a day of thoracic radiation therapy, with or without concurrent chemotherapy.

Cancer therapy can, for example, be provided prophylactically to a subject prior to the development of cancer. Prophylactic administration, also referred to as prevention, is effective to decrease the likelihood that cancer will develop in the subject. The subject in need of prophylactic treatment may be an individual who has or is suspected of having a cancer. In some of variations, the human is at risk of developing a cancer (e.g., a human who is genetically or otherwise predisposed to developing a cancer) and who has or has not been diagnosed with the cancer. As used herein, an “at risk” subject is a subject who is at risk of developing cancer (e.g., a hematologic malignancy). The subject may or may not have detectable disease, and may or may not have displayed detectable disease prior to the treatment methods described herein. An at-risk subject may have one or more so-called risk factors, which are measurable parameters that correlate with development of cancer, such as described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, and environmental exposure. In some embodiments, a human at risk for cancer includes, for example, a human whose relatives have experienced this disease, and those whose risk is determined by analysis of genetic or biochemical markers. Prior history of having cancer may also be a risk factor for instances of cancer recurrence. In some embodiments, provided herein is a method for treating a human who exhibits one or more symptoms associated with cancer (e.g., a hematologic malignancy).

The effectiveness of cancer treatment may be measured by evaluating a reduction in tumor load or decrease in tumor growth in a subject in response to the administration of the modified immune suppressor cells. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

The invention relates to reducing toxic side effects in a subject undergoing cancer treatment. The step of reducing toxic side effects can be conducted after cancer treatment has begun and side effects may have been observed, or can begin at about the same time that cancer treatment begins.

Cancer treatment can result in many toxic side effects. A toxic side effect occurs when treatment damages healthy cells, and can vary depending on the patient and the type of cancer therapy being administered. Examples of toxic side effects from cancer treatment include neutropenia, lymphedema, hair loss, nausea and vomiting, blood clots (e.g., deep vein thrombosis), and inflammation. For a full list of possible side effects from cancer treatment, see for example the National Cancer Institute website entitled “Side Effects of Cancer Treatment.”

In some embodiments, the toxic side effect is inflammation Inflammation is a particularly common toxic side effect when the cancer therapy is immunotherapy, but can also occur in other types of cancer therapy, such as radiotherapy of lung cancer. Immune checkpoint inhibitor therapy can trigger an immune response that causes inflammation to organs in the body of the patient. Inflammation from cancer treatment can occur in the digestive system, the endocrine system (e.g., the pituitary gland, the adrenal gland, the thyroid gland, and the pancreas), the musculoskeletal system, the respiratory system, the skin, the eyes, the heart, the kidney, the liver, and the nervous system Inflammation of the respiratory system includes inflammation of the lungs, including radiation-induced pulmonary pneumonitis.

The invention includes administering an effective amount of a TNF-related apoptosis-inducing ligand (TRAIL, also known as Apo2L) compound to a subject who is undergoing cancer therapy. TRAIL compounds include TRAIL and conservative derivatives or variants of TRAIL, as well as TRAIL-inducing compounds. Examples of TRAIL compounds include ONC201 and TLY012.

TRAIL interacts with death receptors (including TRAIL receptors) on cancer cells and activates apoptotic pathways in cancer cells, but not in healthy cells. Ashkenazi, A., Nat Rev Cancer 2(6): 420-430 (2002). TRAIL shows homology to other members of the tumor necrosis factor superfamily. It is composed of 281 amino acids and has characteristics of a type II transmembrane protein. Three protein sequences for human TRAIL include Accession Number NP_003801 (isoform 1), Accession Number NP_001177871 (isoform 2), and Accession Number NP_001177872 (isoform 3). One variant of TRAIL is Dulanermin, which is a soluble, recombinant formulation of human TRAIL including the extracellular portion of TRAIL (i.e., amino acids 114-281). Other variants of TRAIL include those fused or conjugated with another protein such as serum albumin to increase its circulatory half-life. See Li et al., J. Control. Release, 228:96-106 (2016) and Byeon et al., Bioconjug. Chem., 25:2212-2221 (2014). Additional TRAIL variants include oligomeric TRAIL (Naval et al., Cancers, 11:444 (2019)), viral-bound TRAIL (Wang et al., Signal Transduct. Target. Ther., 5:40 (2020)), and TRAIL tethered to nanoparticles (De Miguel et al., Mol. Pharm., 10:893-904 (2013)). Another TRAIL variant is TLY012, which is an N-terminal PEGylated recombinant human TRAIL. Chae et al., Mol. Cancer Ther. 9, 1719-1729 (2010). Further PEGylated TRAIL analogs are also known. Kim et al., Bioconjug. Chem. 22, 1631-1637 (2011).

Functional-conservative derivatives may result from modifications and changes that may be made in the structure of a polypeptide (and in the DNA sequence encoding it), and still obtain a functional molecule with desirable characteristics (e.g., antiinflammatory effects). Functional-conservative derivatives may also consist of a fragment of a polypeptide that retains its functionality.

Functional-conservative derivatives are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A functional-conservative derivative also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared. Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wisconsin) pileup program, or any of sequence comparison algorithms such as BLAST, FASTA, etc.

In some embodiments, a TRAIL inducing compound is used to decrease toxic side effects. A TRAIL inducing compound is a compound that increases the expression and/or activity of TRAIL in the subject. An example of a TRAIL inducing compound is the small molecule ONC201 (a.k.a. TIC10), the structure of which is shown below. See Allen et al., Mol Cancer, 14:99 (2015). ONC201. A variety of related TRAIL-inducing compounds are described in U.S. Pat. No. 10,172,862, the disclosure of which is incorporated herein by reference. Structure-activity analysis of ONC201 has also identified additional imipridone derivatives capable of inducing TRAIL. Prabhu et al., Neoplasia, 22(12):725-744 (2020).

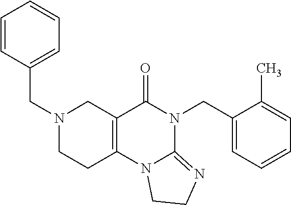 [SMILES: Cc1ccccc1CN1C(=O)C2=C(CCN(Cc3ccccc3)C2)N2CCN=C12]

### Administration and Formulation

The present invention also provides pharmaceutical compositions that include a TRAIL compound as an active ingredient, and a pharmaceutically acceptable carrier or carriers, in combination with the active ingredient.

The compounds can be administered as pharmaceutically acceptable salts. Pharmaceutically acceptable salt refers to the relatively non-toxic, inorganic and organic acid addition salts of the compounds. These salts can be prepared in situ during the final isolation and purification of the compounds, or by separately reacting purified compounds with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions include TRAIL or TRAIL-inducing compounds together with one or more of a variety of physiological acceptable carriers for delivery to a patient, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The TRAIL compounds can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The compounds are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations of the present invention suitable for oral administration may be presented as discrete units such as tablets, troches, capsules, lozenges, wafers, or cachets, each containing a predetermined amount of the active agent as a powder or granules, as liposomes containing the compounds, or as a solution or suspension in an aqueous liquor or non-aqueous liquid such as a syrup, an elixir, an emulsion, or a draught. Such compositions and preparations typically contain at least about 0.1 wt-% of the active agent. The amount of the compound is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

An example has been included to more clearly describe particular embodiments of the invention. However, there are a wide variety of other embodiments within the scope of the present invention, which should not be limited to the particular example provided herein.

### EXAMPLE

It is not sufficient to have effective cancer therapy if there are significant toxicities that impact on quality of life and cause morbidity. A general goal in the field of oncology has been to develop better tolerated therapeutics. Radiation therapy remains as a pillar of cancer therapy with proven efficacy in the local control of cancer as well as patient survival outcomes in multiple tumor types. However, toxicity of radiation towards normal tissues has been a challenge in patient treatment. Other cancer therapeutic agents such as bleomycin or immune checkpoint blockade cause morbidity, can interfere with continuing treatment due to lung inflammation and injury, and ultimately contribute to mortality.

In the lungs the so-called late effects of radiation that involve inflammation and fibrosis are well known to cause morbidity and to limit the use of radiation or other cancer therapies in patients who suffer with symptoms (M. Arroyo-Hernandez et al., BMC Pulmonary Medicine 21, 9 (2021)). While high-dose radiotherapy to the lungs is beneficial in treatment of patients with lung cancer, dose-fractionation is inconvenient and is still associated with severe toxicity (H. G. Yoon et al., Radiat Oncol J 37, 185-192 (2019)). Radiotherapy is often combined with immunotherapy such as immune checkpoint blockade to treat cancer and this increases risk and incidence of serious lung injury (S. Hindocha et al., Front Med (Lausanne) 8, 764563 (2021)). Therefore, strategies are needed to reduce the side-effects of radiotherapy in order to improve the care of patients with cancer. There is also a need to develop radiation countermeasures for use in the unfortunate event of a dirty bomb, nuclear power plant leak or other act of war that exposes people to high levels of radiation (T. J. MacVittie, A. M. Farese, Journal of Radiological Protection 41, S438 (2021)). Such strategies could be employed to achieve radioprotection if the agents are effective when used post-radiation exposure.

We discovered TRAIL death receptor DR5 as a direct transcriptional target of the p53 tumor suppressor protein (R. Takimoto, W. S. El-Deiry, Oncogene 19, 1735-1743 (2000)). The p53 protein is central to the cellular DNA damage response (DDR) that is inflicted by radiation damage. Stabilization of the p53 protein after DNA damage activates genes encoding proteins such as p21(WAF1) that mediate cell cycle arrest and repair of DNA damage (W. S. el-Deiry et al., Cell 75, 817-825 (1993)). Repair of damage is essential for cell survival without cancer. DR5 is involved in the cell death that occurs after treatment of tumors by chemotherapy or radiation. The TRAIL pathway that signals through DR5 is part of the host innate immune system for suppression of cancer and its metastases (B. A. Carneiro, W. S. El-Deiry, Nat Rev Clin Onco117, 395-417 (2020)).

We previously discovered that deletion of the DRS gene in mice is associated with reduced cell death in multiple organs after a lethal dose of radiation (N. Finnberg et al., Mol Cell Biol 25, 2000-2013 (2005)), and that a sub-lethal dose of whole body irradiation causes inflammatory lesions and fibrosis in multiple organs including the lungs of irradiated mice (N. Finnberg, et al., The Journal of Clinical Investigation 118, 111-123 (2008)). This resulted in lethality of irradiated DR5-deficient mice 4-6 months post-irradiation. Histologic analysis of lung tissues from DR5-deficient mice suggested a similarity with what is observed with the late effects of radiation in humans who receive thoracic radiation and develop pneumonitis including deposition of collagen and fibronectin (id.).

We suspected that a primary strategy to rescue from the toxic effects of lung radiation might involve treatment for several months with TRAIL pathway agonists which was not particularly feasible or cost-effective years ago. However, with the availability of new ways of stimulating the TRAIL innate immune pathway (A. V. Jhaveri et al., Cancer Biol Ther 22, 607-618 (2021)), we revisited the prospect of rescue from radiation toxicity. For example, TRAIL-Inducing Compound #10 (TIC10) also known as ONC201 is an anti-cancer agent currently in multiple clinical trials for various tumor types (V. V. Prabhu et al., Neoplasia 22, 725-744 (2020)), while TLY012 is a new PEGylated trimeric TRAIL formulation that has anti-fibrotic properties (J.-S. Park et al., Nature Communications 10, (2019)). Surprisingly, we discovered that short-term treatment with TRAIL pathway agonists for two weeks post-radiation exposure effectively rescued mice that received 20 Gy of thoracic radiation exposure effectively rescued mice that received 20 Gy of thoracic radiation from severe inflammation and lethality.

### Results

Short-term Treatment with TRAIL Pathway Agonists TLY012 or TIC10/ONC201 Protects Wildtype but not DR5-/- Mice from Lung Pneumonitis and Fibrosis Following In Vivo High-Dose Thoracic Ionizing Radiation

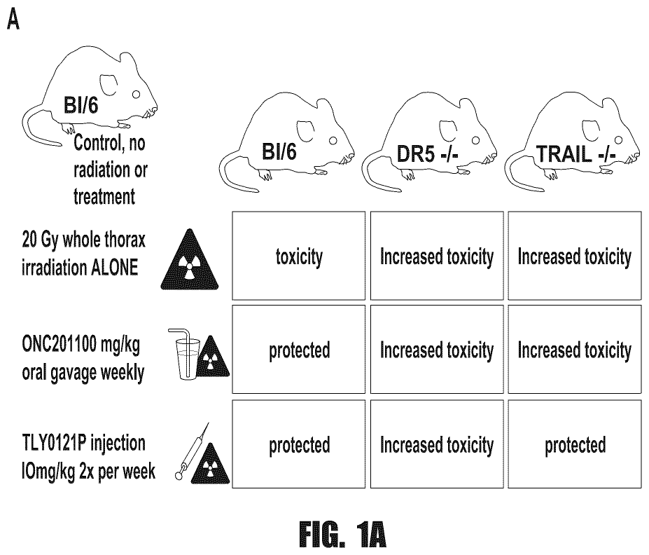

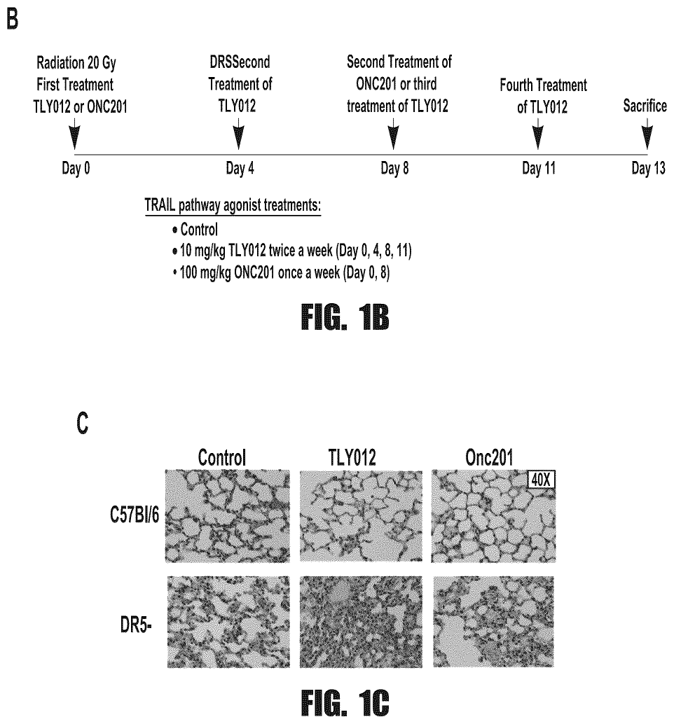

We designed a high dose thoracic radiation experiment in wild-type (WT), DR5-null or TRAIL-null C57B1/6 mice with predicted outcomes as shown in FIG. 1A. Based on our previous work, we hypothesized that DR5-null mice would have more severe radiation-induced pneumonitis and wouldn't be rescued by either a TRAIL ligand or a small molecule TRAIL inducing compound because the receptor for TRAIL (DR5) is deleted in the DR5-null mice (N. Finnberg, et al., The Journal of Clinical Investigation 118, 111-123 (2008)). We also hypothesized that a TRAIL formulation such as TLY012 would rescue pneumonitis in TRAIL-null mice whereas a TRAIL gene-inducing compound such as TIC10/ONC201 would not because the TRAIL gene is deleted in TRAIL-null mice.

However, before embarking on the planned experiment, we decided to perform a preliminary, short-term treatment feasibility experiment to ensure some mouse survival after 20 Gy of thoracic irradiation (FIG. 1B). The preliminary experiment was performed with WT and DR5-null mice that were irradiated with 20 Gy, and this was followed by either weekly doses of TIC10/ONC201 (100 mg/kg for total of two doses) or twice a week doses of TLY012 (10 mg/kg for total of four doses) for 2 weeks. As shown in FIG. 1C, we observed qualitative evidence of protection from pneumonitis in WT mice treated with either TLY012 or ONC201 versus control (20 Gy-irradiated mice that did not receive either TLY012 or ONC201). It was clear that the lung inflammation in the irradiated mice was much worse in DR5-null mice regardless of whether they were treated with TLY012 or ONC201 (FIG. 1C). It was surprising that few doses of either TRAIL pathway agonist appeared effective in protecting against the observed radiation pneumonitis. The lack of protection of DR5-null mice or in fact the appearance of a more severe phenotype is consistent with our previous observations (N. Finnberg, et al., The Journal of Clinical Investigation 118, 111-123 (2008)). No obvious toxicities were noted in duodenum, liver or heart.

Rescue of Irradiated Wild-Type or TRAIL-/- Mice but not DR5-/- Mice from Radiation Pneumonitis by TLY012

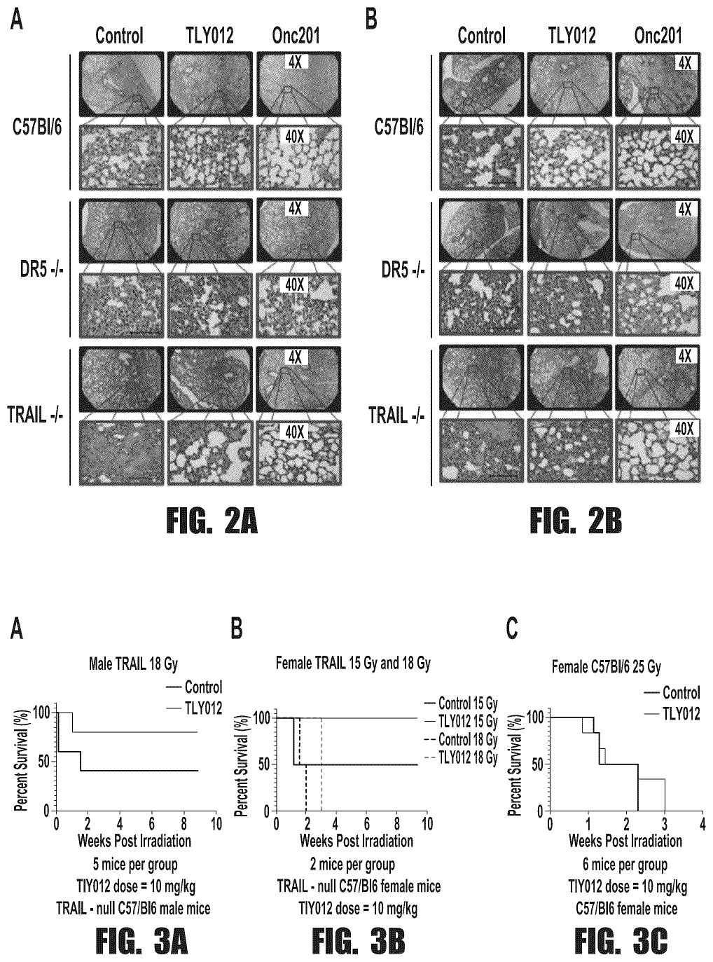

We tested the predictions that TRAIL-/- mice would be rescued by TRAIL (TLY012) but not by TIC10/ONC201 (FIG. 1A). As predicted, short-term treatment with TLY012 over two weeks rescued 20 Gy irradiated (thoracic irradiation) male (FIG. 2A) or female (FIG. 2B) mice while ONC201 did not. This is consistent with the notion that if the TRAIL gene is not present, then ONC201 would not be expected to increase expression of the gene and rescue the pneumonitis. To our knowledge, the results in FIG. 2A, B demonstrate for the first time not only the severe inflammation in 20 Gy-irradiated TRAIL-null lungs, but also their rescue by a TRAIL formulation (TLY012). In FIG. 2A, B we did not observe rescue of DR5-/- 20 Gy-irradiated lungs by either TLY012 or ONC201. We noted surprisingly that the severity of radiation-induced pneumonitis appeared worse in female versus male mice. Prior studies have either seen more toxicity in male rats or patients (M. S. Rahi et al., Clin Pract 11, 410-429 (2021)). In the case of the patients, it may be that more men had lung cancer and were treated with radiation.

### Cytokine Alterations in TLY012-Treated Mice

We examined patterns of cytokines in WT C57B1/6, DR5-/- and TRAIL-/- mice that were irradiated and either not subsequently treated (control) or that were treated with TLY012 or ONC201. We observed that IGF1 was upregulated by ONC201 while TLY012 increased CXCL1, IL6, GDF-15, IL6, MMP-8, and CCL3/MIP- lalpha. An increase in FGF basic was noted with either TLY012 or ONC201. Prior work has implicated a range of other cytokines including TGF-beta, IL6, TNF and other inflammatory cytokines (A. Lierova et al., J Radiat Res 59,709-753 (2018)). We believe our results are shedding light upon factors whose role is brought out by the effective preventative agents targeting activation of the TRAIL pathway.

TLY012 Protects from Lethality of Radiation Pneumonitis in TRAIL-/- Mice

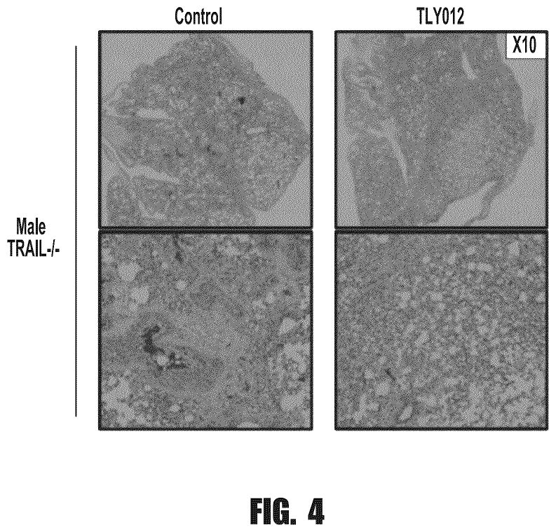

We investigated whether TLY012 could rescue mice from the lethality of thoracic irradiation (FIG. 3). We observed rescue from lethality of TRAIL-/- male C57B1/6 mice with 10 mg/kg dosing with TLY012 after a dose of 18 Gy of chest irradiation (FIG. 3A). For female TRAIL-/- C57B1/6 mice we needed to reduce the radiation dose to 15 Gy to observe protection from lethality (FIG. 3B). There was no protection from lethality by TLY012 of WT female C57B1/6 mice after an extremely high single dose of 25 Gy (FIG. 3C).

### Reduced Inflammation, and DNA Damage in Lung-Irradiated Versus TLY012-Rescued Mice

Examination of lung tissue in irradiated C57B1/6 mice revealed that while TLY012 or ONC201 could rescue mice from radiation-induced pneumonitis (FIG. 2), there was no difference in T-cell infiltration as judged by the pan T-cell marker CD3-epsilon or the DNA double strand break marker gamma-H2AX within what remained as normal lung or what was inflamed lung with pneumonitis. What was clear was that overall, there were fewer areas of pneumonitis in the rescued mice (FIG. 2) but no differences in marker expression either in the analyzed healthy tissue or the inflamed tissue in control versus treated mice. We observed no differences in NFkB or proliferation between irradiated controls and irradiated rescued lung tissues. There was less collagen in rescued lungs. Thus overall, there was less inflammation, DNA damage and collagen deposition in rescued lungs although in any residual inflamed tissues there was no apparent difference in those regions.

Decrease in Collagen Production in Mice Receiving 18 Gy Thoracic Radiation Dose Treated with TLY012

In order to examine the late term effects of thoracic radiation, we designed an experiment where the single radiation dose was lowered to 18 Gy in male TRAIL-/- mice. For 22 weeks post radiation, mice were either treated with 10 mg/kg of TLY012 twice a week or remained control. At 22 weeks when mice reached criteria for euthanasia, they were sacrificed and lung tissue was harvested. Lung tissue slides were stained with Masson's trichrome and imaged at 10X on an Olympus VS200 slide scanner. Upon analysis, there is significantly more light blue staining seen in the control mice compared to the TLY012 treated mice. The decrease in collagen deposition observed in the TLY012 treated mice suggests that there was a decrease in inflammation and a rescue from late effects of radiation pneumonitis.

Alteration in mRNA Levels of Genes Related to Inflammation and Immune Response

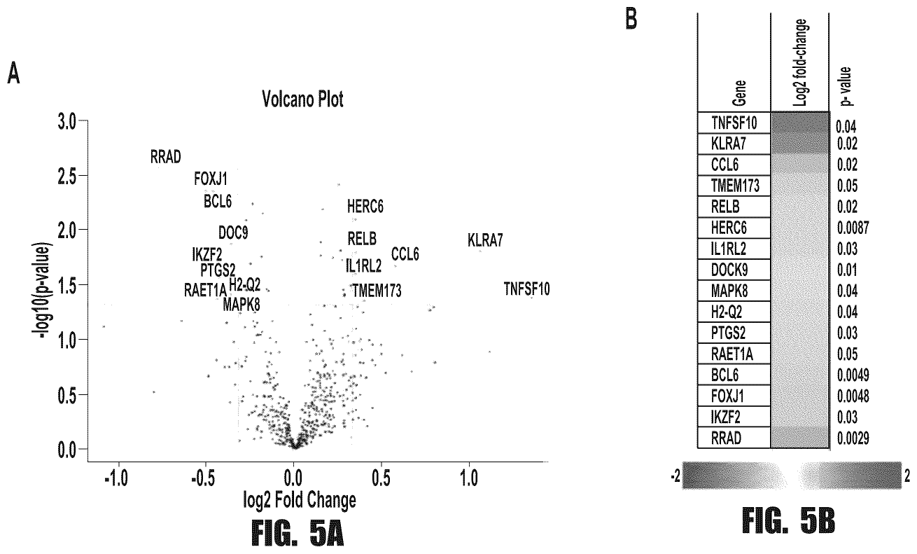

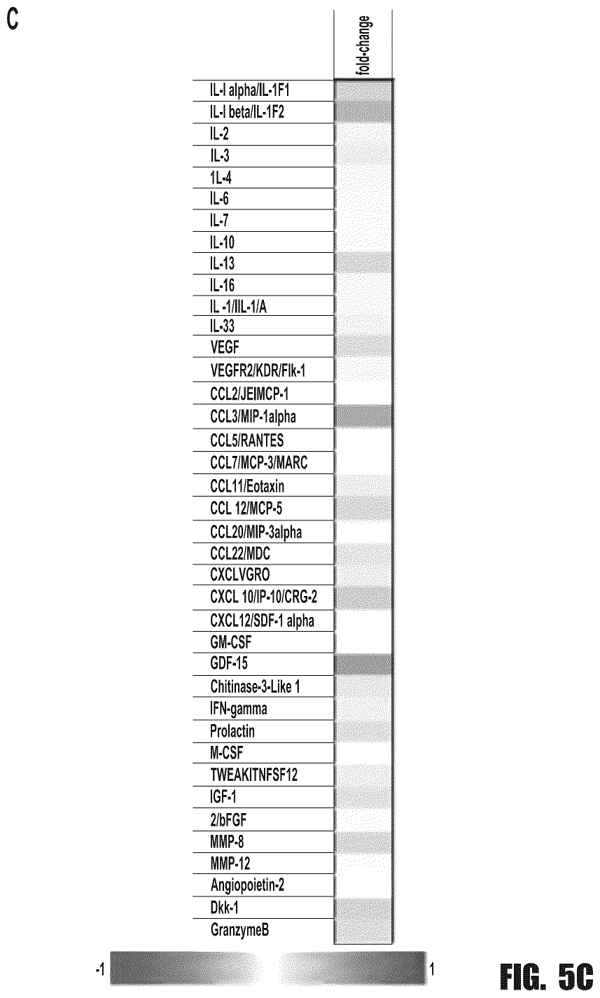

In order to determine the change in immune response between mice treated with TLY012 and controls after a single dose of 20 Gy thoracic x-ray irradiation, a NanoString PanCancer Immune Profiling panel was used to analyze mRNA from mouse lung tissue. It was found that 16 genes had statistically significant differential expression between the TLY012 treated group and the control group. TNFSF10, KLRA7, CCL6, TMEM173, RELB, HERC6, and IL1RL2 were among the top upregulated differentially expressed genes (DEGs) in the TLY012 treated group relative to the control group, while DOCKS, MAPK8, H2-Q2, PTGS2, RAET1A, BCL6, FOXJ1, IKZF2, and RRAD are among the top downregulated DEGs (FIG. 5A, B). NanoString pathway changes between the TLY012 treatment group and the control group were also examined. It was found that there were significant increases in “Antigen Processing” and “Interferon” pathways. When treatment TLY012 treated group and control group were further separated into male and female cohorts, it was found that there were significant increases in the “Antigen Processing”, “MHC”, and “Dendritic Cell Functions” pathways and a decrease in the “Basic Cell Functions” pathway in the female TLY012 treated mice.

In addition to the NanoString PanCancer Immune profiling panel, serum cytokine analysis was performed. The cytokines that had the greatest differential expression in the TLY012 treated mice was a decrease in CCL3/MIP-lalpha, GDF-15 as well as an increase in IL-1beta (FIG. 5C).

Orthotopic Breast Tumor-Bearing Immune-Competent Mice Receiving a 20 Gy Thoracic Radiation Dose were Protected from Pneumonitis While Showing Complete Elimination of Tumors

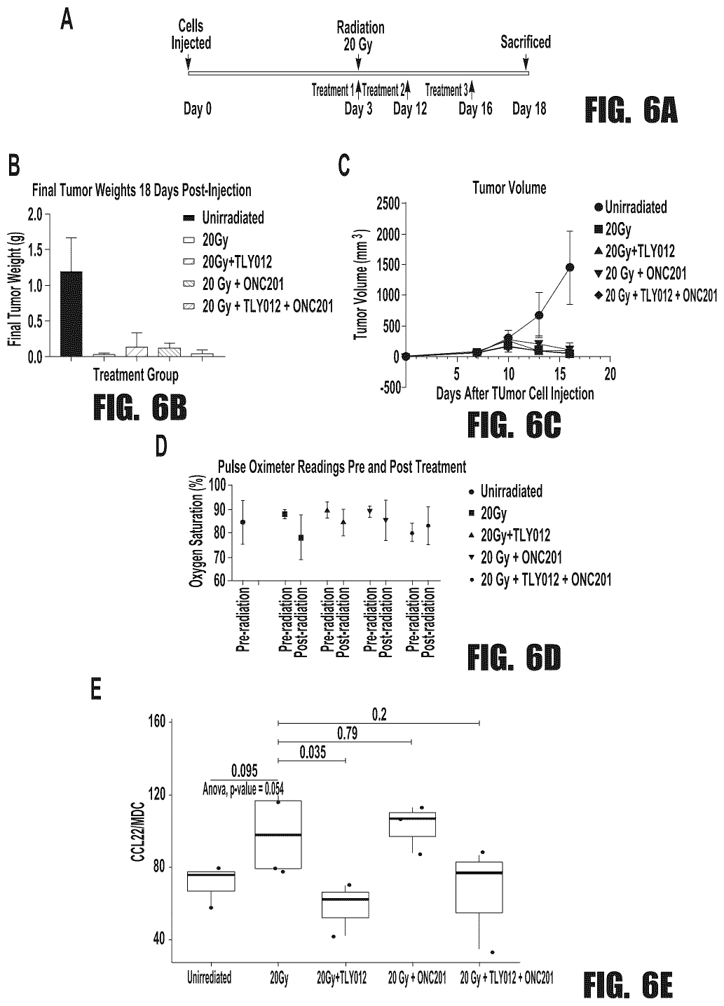

We set up an experiment to determine whether TLY012, ONC201 or the combination used to prevent radiation pneumonitis might in any way interfere with the anti-tumor effect of radiation to an orthotopically implanted breast cancer in immune competent mice. We did not expect either ONC201, TLY012 or the combination to block anti-tumor efficacy given our prior work (M. D. Ralff et al., Oncotarget 11, 3753-3769 (2020)), but needed to formally show this in the radiation pneumonitis context. We injected mouse breast cancer e0771 cells orthotopically into mammary fat pad #2 of immune-competent C57B1/6 mice and subsequently administered 20 Gy of radiation to the chest on day 9. Mice received TLY012, ONC201, the combination or no further treatment on days 9, 12, and 16 and then mice were sacrificed on day 18 (FIG. 6A). We observed an anti-tumor effect of radiation with no evidence that ONC201, TLY012 or their combination reduced efficacy of treatment (FIG. 6B, C). We detected reduced oxygen saturation in treated mice versus unirradiated mice and this was partially rescued by either TLY012 or ONC201 versus irradiated mice that received no further treatment (FIG. 6D). Rescue of mice from radiation induced pneumonitis appeared to be more potent by TLY012 than with ONC201. The combination of TLY012+ONC201, while effective, did not appear to improve the protection from pneumonitis under these experimental conditions. We observed reduction in CCL22/MDC levels in TLY012-treated irradiated tumor-bearing mice. CCL22 (FIG. 6E), a macrophage-derived chemokine has been previously associated with radiation pneumonitis and pulmonary fibrosis (T. Inoue et al., European Respiratory Journal 24, 49 (2004)).

In vivo Micro-Computed Tomography (XT) Scans of Mouse Lung Show from Radiation Induced Lung Injury and Fibrosis when Treated with TLY012.

In order to determine the effects of radiation on the mice in vivo, micro-computed tomography scans were taken of TRAIL-/- female mice that were unirradiated and two weeks-post one single thorax x-ray irradiation dose of 15 Gy on TRAIL-/- female mice with and without twice weekly TLY012 treatment (n=2/treatment/group). Individual μCT image slices were collected as well as 3D reconstruction of the lungs during the inhale and exhale duration of the breathing cycle. In the μCT mouse lungs expand more upon inhalation in the unirradiated and mice that were irradiated with 15 Gy and treated with TLY012 compared to the 15 Gy irradiated control. The distance between the heart and the esophagus is visually greater upon maximum inhale images in the

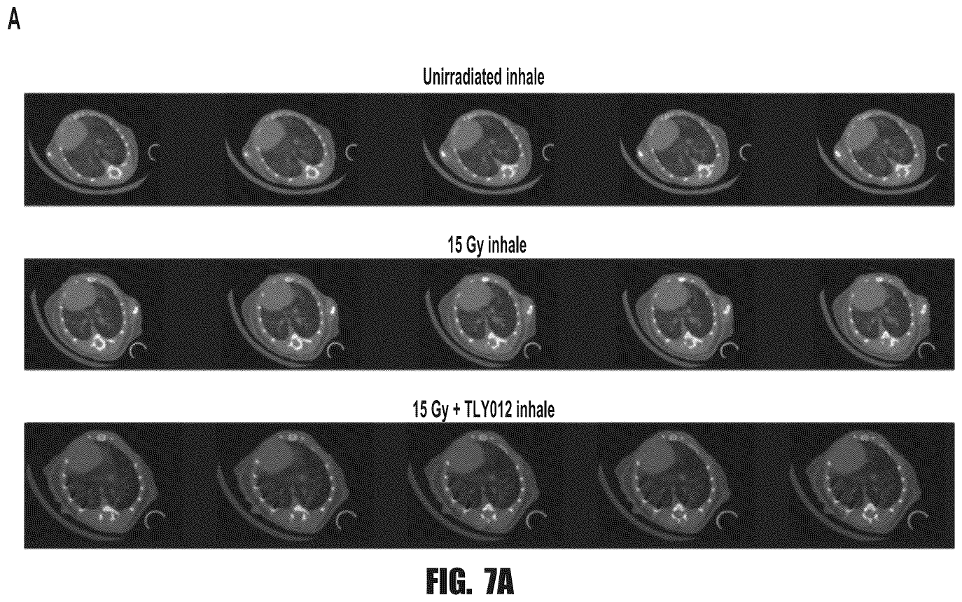

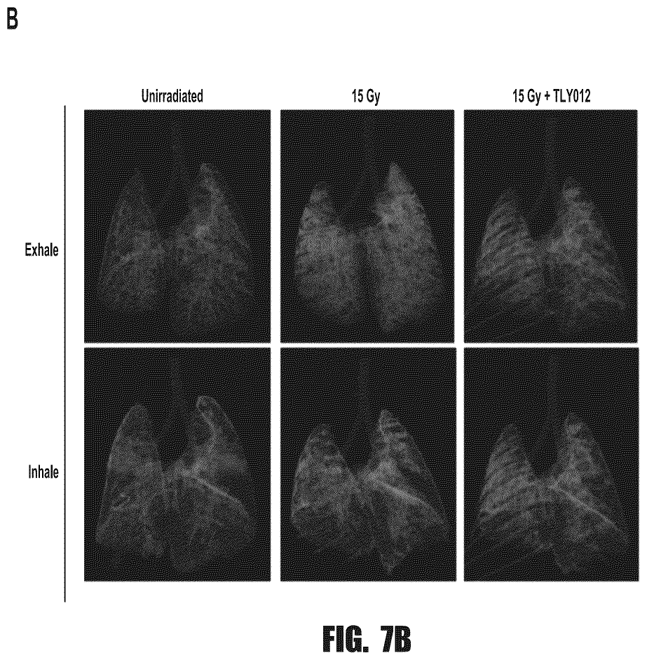

TLY012 treated group as compared to the 15 Gy group, demonstrating the lungs were able to expand more (FIG. 7A). In the 3D reconstruction, when all the images were subjected to the same opacity conditions the unirradiated control and the 15 Gy irradiated group treated with TLY012 are overall clearer and have more visible airway space, particularly during the exhale portion of the breathing cycle (FIG. 7B). These observations are consistent with post-mortem findings through H&E and immunohistochemistry.

### DISCUSSION

The impact of this work is in the prevention of the lethality or other severe adverse consequences of therapeutic or unanticipated radiation injury in the lungs such as pneumonitis and fibrosis by short-term treatment with innate immune TRAIL pathway agonists. Although we initially discovered the connection between the TRAIL death receptor signaling pathway and radiation pneumonitis nearly two decades (N. K. Finnberg, et al., Cancer Research 66, 184-184 (2006)), not until recently was it feasible to test whether this could be addressed through pharmacological interventions. It was surprising that short-term treatment with either TRAIL pathway agonist TLY012 or TIC10/ONC201 for two weeks prevented pneumonitis or lethality. This has implications for management of lung toxicity from radiation or in the area of radiation counter-measures. Preventing acute lung inflammation prevents delayed effects of radiation on the lungs and chronic lung injury.

One of the limitations of this study is that the lungs were not re-inflated post-mortem. We conducted a separate study comparing the H&E of inflated lungs versus non-inflated lungs of unirradiated mice and concluded that the alveolar border thickness was not greatly impacted by inflation. Consistently across all of our models the lungs were not inflated but the lung inflammation and its rescue were obvious and functionally documented in vivo.

Another limitation of this study is the number of mice that were used in each experiment. While each individual experiment only included a small number of mice, we continued to observe that TLY012 mitigated radiation pneumonitis across multiple experiments in male and female mice.

While there were statistically significant changes in lung tissue in mRNA levels of genes related to immune response but no statistically significant fold-change in the cytokine levels of relative inflammatory markers in serum. The lack of significance in cytokines could be due to the fact that the cytokine levels were measured from serum levels and not from bronchoalveolar lavage (BAL). The mRNA was extracted from lung tissue while cytokine levels were measured from peripheral blood serum levels. In future experiments BAL could be tested to determine if differential cytokine activity is more prominent in the lungs. It should be noted that serum samples were taken 3 days after last administration of TLY012.

We note and emphasize that there was no difference in DNA damage (gamma-H2AX) or T-cell inflammation (CD3-epsilon) in the irradiated lungs in healthy or inflamed areas of the lungs regardless of whether they were rescued by TRAIL pathway activation or not. However, rescued mice had far fewer areas of pneumonitis, physiologically relevant increase in oxygen saturation, and evidence of rescued alveolar border thickness by histological examination of the lungs. The prevention of the pneumonitis by the TRAIL innate immune pathway agonists has no detrimental effects on the use of therapeutic radiation with regard to anti-tumor efficacy. Our results provide important clues as to alterations in cytokine biomarkers such as CCL22 or others that were impacted by TLY012.

While H&E images of the lungs showed a reduced inflammatory phenotype in mice treated with TLY012, changes in gene and cytokine expression showed decrease in inflammation as well. Genes related to a decrease in inflammatory response were increased in TLY012-treated mice. Killer cell lectin-like receptor 7 (KLRA7) is expressed by mature NK cells in mice (G. h. Ran et al., Signal Transduction and Targeted Therapy 7, 205 (2022)). Re1B, a member of the NF-kappaB/Re1B family is known to act as a transcription suppressor in fibroblasts which limits the expression of proinflammatory mediators which had a significantly increased expression in TLY012 treated mice (Y. Xia et al., Molecular and Cellular Biology 19, 7688-7696 (1999)). Transmembrane protein 173 (TMEM173) encodes protein stimulator of interferon genes (STING), a key player in host defense against damaged cells. TMEM173 plays an important role in normal pulmonary function as it was found that loss-of-function TMEM173 alleles in humans lead to pulmonary fibrosis (S. Patel, L. Jin, Genes & Immunity 20, 82-89 (2019)). HERC6 mediates ISGylation, which is involved in DNA repair and autophagy (C. Villarroya-Beltri, et al., Journal of Cell Science 130, 2961-2969 (2017)). Interleukin 1 receptor like 2 (IL1RL2), which has an anti-inflammatory effect and can regulate macrophage function was also upregulated in mice treated with TLY012 (S. Kurose et al., Physiological Reports 11, e15581 (2023)). All of these findings suggest that treatment with TLY012 can prevent the inflammation and fibrotic effects caused by radiation.

Decreased expression of genes related to a proinflammatory response were also observed in mice that were treated with TLY012. Dedicator of cytokinesis 9 (DOCKS), which is thought to play a role in in immune disorders, was shown to have a decrease in mRNA expression in mice treated with TLY012 (K. Namekata et al., Journal of Biological Chemistry 295, 6710-6720 (2020)). Mitogen-activated protein kinase 8 (MAPK8), also referred to as c-Jun amino terminal kinase (JNK) is activated in response to various cellular stresses and promotes production of pro-inflammatory and pro-fibrotic molecules was also significantly decreased in treated mice (K. Grynberg, et al., Frontiers in Physiology 8, (2017)). Prostaglandin-endoperoxide synthase 2 (PTGS2), also known as COX2 is an enzyme that is responsible for causing chronic inflammation (C. Chen, Nature Chemical Biology 6, 401-402 (2010)). Retinoic acid early transcript 1 alpha (RAET1A) functions as a stress-induced ligand for NKG2D receptor which is expressed on cytotoxic immune cells was downregulated in TLY012 treated mice, meaning the control mice were in a greater state of stress (A. Sagiv et al., Aging 8, 328-344 (2016)). Another indicator of inflammation, B cell leukemia/lymphoma 6 (BCL6), was decreased in treated mice (B. Zhu et al., Proceedings of the National Academy of Sciences 116, 11888-11893 (2019)).

There remain open questions and research directions of interest. These include further mechanistic studies in the role of the cytokines and chemokines in the pathogenesis of pneumonitis or rescue from it. There are major questions about cross-talk between the TRAIL pathway and the TGF-beta pathway, Gas/STING, and other cytokine responses previously linked to radiation pneumonitis. Our findings may have relevance to other lung injury models such as adult respiratory distress syndrome, idiopathic pulmonary fibrosis, immune checkpoint blockade pneumonitis, COVID-19 pneumonitis, or toxicities of other chemotherapy agents such as bleomycin. Clearly combinations with other therapies such as steroids or TGF-β pathway inhibitors could be further studied and more work needs to be done to understand differences between males and females in how they develop lung inflammation and fibrosis and why the phenotype in females is more severe in DR5-/- and TRAIL-/- mice. Further studies need to address protection of other organs including bone marrow, GI tract, or the brain from toxic effects of a range of radiation doses. More work needs to determine how late in time TRAIL pathway agonists could impact on protection from radiation post-exposure including in combination with other agents. Our preliminary results suggest some rescue is possible even at 48 hours after irradiation and treatment by TLY012. Our findings have translational relevance by suggesting clinical investigation of TRAIL innate immune pathway in toxicity of radiation or other cancer therapeutics, other inflammatory lung conditions and have implications for the field of radiation countermeasures.

### MATERIALS AND METHODS

### Bioassays

Animal studies were carried out at Brown University facilities with approval from Brown University IACUC. C57B1/6 mice (Taconic), mice with bi-allelic loss of TRAIL Death Receptor-(DR5-/-) in a C57B1/6 background, and mice with bi-allelic loss of TRAIL (TRAIL -/-) in a C57B1/6 background were given a single whole-thorax x-ray (Philips RT250) irradiation dose of 20 Gy with shielding of other organs. 8-15-week-old male and female mice of each genotype were used. Starting one hour before irradiation, mice were treated with either 10 mg/kg of TLY012 (D&D Pharmatech) by intraperitoneal (IP) injection or 100 mg/kg of ONC201/TIC10 (Chimerix/Oncoceutics) by oral gavage (PO) or a control gavage consisting of 20% Cremophor® EL (Sigma #238470) and 70% Dulbecco's phosphate buffered saline solution (Cytiva SH30264.02) per volume via PO (n=2/gender/treatment). Treatment was continued for two weeks, where TLY012 was administered twice a week and ONC201 was administered once a week. For each genetic background, two mice received a control PO, two mice received TLY012 IP, and two mice received ONC201 PO. Mice were weighed twice a week, and 13 days post-irradiation all mice were euthanized. Organs were harvested from the mice and 600 μL of blood was collected via cardiac puncture for serum cytokine analysis. Organs were preserved in 10% formalin then embedded in paraffin and sectioned at a distance of 5 μm. Hematoxylin and eosin (H&E) stained slides of the lungs, heart, liver, and duodenum were imaged at 4× and 40× magnifications on a Nikon Y-THM Multiview Main Teaching Unit microscope using a Diagnostic Instruments, Inc. model 18.2 color mosaic camera paired with SPOT Basic version 5.3.5 software. Survival experiments were conducted using male TRAIL-/- mice treated with either TLY012 or control exposed to a whole-thorax radiation dose of 18 Gy (n=5/treatment) as it was determined any dose above 18 Gy caused too great of radiation toxicity in order to allow long term survival (FIG. 3A). TRAIL-/- female mice were also subjected to a survival study with a single whole-thorax radiation doses of 15 Gy or 18 Gy treated with the same regimen of TLY012 or control (n=2/radiation dose/treatment) (FIG. 3B). In order to determine the lethal dose of radiation for C57B1/6 mice, female C57B1/6 mice were treated with a single dose of whole-thorax irradiation of 25 Gy in a survival study where they were treated with either TLY012 or remained control (n=6/treatment) (FIG. 3C).

### Tumor Bearing Model

C57B1/6 female mice (Taconic) were injected with 100,000 cells of murine breast cancer (e0771) in the right second mammary fat pad on day 0 of the experiment. On day 9, tumors were greater than 4 mm in diameter and mice were grouped into control, treatment of 10 mg/kg of TLY012 twice a week, 100 mg/kg of ONC201 once a week, or a combination of both drugs. Each group (n=3) also received a single whole-thorax x-ray irradiation dose of 20 Gy with shielding of other organs. One group (n=3) received no treatment and no radiation. The long axis and short axis of the tumors were measured using calipers and recorded twice a week. On day 18 all mice were euthanized at a humane endpoint, which was defined by 20% weight loss and/or a tumor volume of 2000 mm³ (˜10% of body weight). Organs and tumors were harvested from the mice and 600 μL of blood was collected via cardiac puncture for serum cytokine analysis. Once tumors were extracted, they were weighed and measured to determine final volume and weight. Pulse oximeter readings were taken initially before radiation treatment and before sacrifice 9 days post-radiation (MouseSTAT Jr. Pulse Oximeter, Kent Scientific). When mice were under anesthesia the sensor of the MouseSTAT Jr. was placed on the hind right paw for 30 seconds. The highest and lowest values shown by the pulse oximeter during this time were recorded and then averaged together. The mean of all the averages per treatment group was then calculated.

### DR5 Agonist

8-13-week-old male TRAIL-/- mice were given a single whole-thorax x-ray irradiation dose of 20 Gy with shielding of other organs. An hour before radiation, mice were treated with 100 μg anti-DR5 mAb (clone MD5-1; BioXCell) or given 100 μg isotype control of polyclonal Armenian hamster IgG (BE0091; BioXcell) via IP injection (n=3/treatment). Mice were treated once a week for two weeks and sacrificed on day 13-post irradiation. Lungs were harvested and preserved in 10% formalin then embedded in paraffin and sectioned at a distance of 5 μm. H&E-stained slides of the lungs were imaged at 20× magnifications.

### 48-Hour Post-Radiation TLY012 Treatment

8-week-old female C57B1/6 mice (Taconic) were given a single whole-thorax x-ray irradiation dose of 20 Gy with shielding of other organs. 48-hours after administration of radiation, mice were grouped into control or treated with 10 mg/kg of TLY012 twice a week (n=4/treatment). Mice were weighed twice a week, and then euthanized on day 13 post-irradiation. Lungs were harvested and preserved in 10% formalin then embedded in paraffin and sectioned at a distance of 5 μm. H&E-stained slides of the lungs were imaged at 20× magnification.

### Cytokines

All mouse serum samples were analyzed by a custom R&D systems Murine Premixed Multi- Analyte Kit (R&D Systems, Inc., Minneapolis, MN). The panel was run on a Luminex 200 Instrument (Luminex Corporation, Austin, TX) according to the manufacturer's instructions. Murine serum levels of Angiopoietin-2, BAFF/BLyS/TNFSF13B, CCL2/JE/MCP-1, CCL3/MIP-1 alpha, CCL4/MIP-1 beta, CCL5/RANTES, CCL7/MCP-3/MARC, CCL11/Eotaxin, CCL12/MCP-5, CCL20/MIP-3 alpha, CCL21/6Ckine, CCL22/MDC, Chitinase 3-like 1, CXCL1/GRO alpha/KC/CINC-1, CXCL10/IP-10/CRG-2, CXCL12/S DF- 1 alpha, Dkk-1, FGF basic/FGF2/bFGF, GDF-15, GM-CSF, Granzyme B, IFN-gamma, IGF-I/IGF-1, IL-1 alpha/IL-1F1, IL-1 beta/IL-1F2, IL-2, IL-3, IL-4, IL-6, IL-7, IL-10, IL-13, IL-16, IL-17/IL-17A, IL-27, IL-33 M-CSF, MMP-3, MMP-8, MMP-12, Prolactin, TWEAK/TNFSF12, VEGF, and VEGFR2/KDR/Flk-1 were measured. Analyte values were reported in picograms per milliliter (pg/mL). TGF-β samples were run using a 1:15 dilution factor, as suggested by the manufacturer. Latent TGF-β was activated in samples using 1 N HCl and samples were neutralized with 1.2 N NaOH/0.5 M HEPES. Samples were assayed immediately after neutralization.

### Immunohistochemistry (IHC)

Paraffin embedded sections of tissue were deparaffinized in xylene and rehydrated through graded ethanol solutions to phosphate-buffered saline (PBS). Heat-induced antigen retrieval (in 0.01 M citrate buffer; pH 6.0), endogenous peroxidase blocking (3% H202), and blocking were done using a standard protocol. Diluted primary antibody (CST #8242 1:800; CST #99940, 1:150; CST #9718,1:500; Abcam ab107099, 1:X; Leica P53-CM5P-L 1:200) was applied and slides were incubated at 4° C. overnight. Slides were then washed with PBS and incubated at room temperature for one hour with appropriate secondary antibodies (Vector Laboratories MP-7401, MP-7402). Slides were developed with diaminobenzidine substrate (Vector Laboratories SK-4100) and counterstained with hematoxylin (Richard Allen Scientific). Slides were scanned (Olympus VS200) and representative brightfield images were taken and analyzed with OlyVIA software.

### NanoString Assay

9-week-old male and female TRAIL -/- mice were given a single whole-thorax x-ray irradiation dose of 20 Gy with shielding of other organs. One hour before radiation mice were grouped into control or given 10 mg/kg of TLY012 via IP injection (n=3/gender/treatment). Mice were treated and weighed twice a week up until 11 days post-radiation when all mice were euthanized.

Rodent sacrifice was performed following anesthetization with 100 mg/kg ketamine and 10 mg/kg xylazine administered intraperitoneally. ˜400-600 μL blood was first collected throughcardiac puncture with a 26G ⅝″ needle through the intercostal space into the left ventricle. Following cervical dislocation, the inferior vena cava (IVC) and descending abdominal aorta were severed prior to bilateral thoracotomy to expose the heart and lungs. 1 mL of 2 mg/mL EDTA in PBS was injected through the right ventricle to perfuse the lungs, after which the lungs were excised, washed further in PBS, submerged in RNAlater (Sigma-Aldrich, #R0901, Missouri, USA), and flash frozen in liquid nitrogen.

Frozen lung tissue was stored long-term at −80° C. Thawing was performed over 16 hours at 4° C. Tissue homogenization was performed with Precellys Lysing Kit (Cayman Chemical Company, #16859, Michigan, USA) and a Bulley Blender Homogenizer (Next Advance, #G14-G15) prior to RNA extraction with QlAgen RNeasy kit (Qiagen, #74104, Hilden, Germany) and cleaned up with QIAgen RNeasy MinElute Cleanup kit (Qiagen, #74204, Hilden, Germany). Quality and concentration were verified by Nanodrop. Extracted RNA samples were gene expression profiled by NanoString nCounter PanCancer Immune Profiling Panel (NanoString Technologies, #XTCSO-MIP1-12, Seattle, WA) according to the manufacturer's instructions.

Nanostring data was analyzed in nSolver Advanced Analysis Software and ROSALIND. Raw data was uploaded to nSolver for automated normalization, background subtraction, and quality control (QC) check. All samples passed QC. Control mice and mice treated with TLY012 lung samples were used to construct two groups to which an unpaired t-test was run to generate the data in the volcano plot that was created in ROSALIND (FIG. 5). Differential expression was determined with p-values and Benjamini-Yekutieli adjusted p-values. Pathway scores are generated in nSolver as a summarization of expression level changes of biologically related groups of genes. Pathway scores are derived from the first Principal Components Analysis (PCA) scores (1st eigenvectors) for each sample based on the individual gene expression levels for all the measured genes within a specific pathway. The cell type score itself is calculated as the mean of the log2 expression levels for all the probes included in the final calculation for that specific cell type.

### In Vivo MicroCT Imaging

For μCT imaging, all mice were imaged in a SkyScan 1276 in vivo microCT scanner (Bruker, Kontich, Belgium) under isoflurane at 41 μm voxels with a 0.7° rotation step in supine position. Mice that underwent μCT of the lungs consisted of TRAIL-/- females that were unirradiated, and irradiated at 15 Gy with and without treatment of 10 mg/kg of TLY012 twice a week (n=2/group). Mice underwent μCT scans 13 days post irradiation. Image-based respiratory gating was used to sort images into gates based on specific time points of the breathing cycle to minimize motion artifact in the final reconstruction. For 3D reconstruction, Dataviewer version 1.5.4 (Bruker) was used to sort the CT images using the function “Listmode Scan” grouping the images in each bin where bin 0=maximum exhale and bin 3=maximum inhale (empty views <10%). Once the images were sorted, the listmode dataset was opened in NRecon software (Bruker) where the ROI undergoes 3D reconstruction. Reconstructed images were then analyzed in CTAn software (Bruker) where the volume of the lungs was calculated during max inhale and max exhale. The plugins in the custom processing page were used to create an ROI that separated the lungs from the rest of the image. 3D reconstructed images of the lung only were then opened in CTvol software (Bruker) where the color (red=69%, green=50%, blue=50%) and the opacity (7%) of the lung was changed.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of reducing toxic side effects in a subject undergoing cancer treatment, comprising administering an effective amount of a TRAIL compound to the subject.

2. The method of claim 1, wherein the toxic side effect is inflammation.

3. The method of claim 2, wherein the inflammation is radiation-induced pulmonary pneumonitis.

4. The method of claim 1, wherein the cancer treatment is radiation treatment.

5. The method of claim 1, wherein the subject has been diagnosed with lung cancer.

6. The method of claim 1, wherein the subject is human.

7. The method of claim 1, wherein the TRAIL compound is a TRAIL inducing compound.

8. The method of claim 1, wherein the TRAIL compound is ONC201.

9. The method of claim 1, wherein the TRAIL compound is TRAIL protein or a TRAIL variant.

10. The method of claim 1, wherein the TRAIL compound is TLY012.
